(19) United States
(12) Patent Application Publication
(10) Pub. No.: US 2016/0222334 A1
(43) Pub. Date: Aug. 4, 2016

(54) FERMENTATION MONITORING DEVICE AND METHOD FOR MONITORING A FERMENTATION PROCESS

(71) Applicant: ENHEART INC., Hsinchu City (TW)

(72) Inventors: Tsan-Hao YANG, Kaohsiung City (TW); Ching-Yen CHANG, New Taipei City (TW); Yu-Jui CHEN, Taipei City (TW); Yu-Wen HUANG, Tainan City (TW)

(21) Appl. No.: 14/957,229

(22) Filed: Dec. 2, 2015

(30) Foreign Application Priority Data
Feb. 4, 2015 (TW) 104103728

(51) Int. Cl. C12M 1/24; C12M 1/00; C12M 1/12; C12M 1/34

(52) U.S. Cl. CPC C12M 23/08; C12M 41/40; C12M 41/14; C12M 41/34; C12M 41/26; C12M 41/32; C12M 37/04

(57) ABSTRACT

A fermentation monitoring device includes a cap body for sealing an opening of a bottle in which a fermentation process is to occur, a detecting unit and a processor. The detecting unit is configured to continuously monitor an environment parameter in a fermentation space defined by the cap body and the bottle. The processor is operable to: estimate when the fermentation process is to be completed, based on a value of the environment parameter; determine whether the value of the environment parameter is within a predetermined range; and generate an alert response when the determination is affirmative.

### CROSS-REFERENCE TO RELATED APPLICATION

This application claims priority of Taiwanese Application Ho. 104103728, filed on Feb. 4, 2015.

### FIELD

The disclosure relates to a fermentation monitoring device and a method tor monitoring a fermentation process that is to be implemented by the fermentation monitoring device.

### BACKGROUND

A fermentation process typically involves putting certain ingredients into a sealed space, in order to produce a desired product (e.g., wine, beer, yogurt, etc.). In the fermentation process, chemical reactions occur and convert specific molecules (e.g., carbohydrate) into other molecules (e.g., alcohol).

During the fermentation process, one or more environment parameters inside the sealed space may need to be closely monitored. Moreover, a time to complete the fermentation process may vary widely based on the desired product and the condition of the sealed space.

### SUMMARY

One object of the disclosure is to provide a fermentation monitoring device that is configured to automatically monitor a fermentation process occurring in a bottle.

According to the disclosure, the fermentation monitoring device includes a cap body, a detecting unit and a processor.

The cap body is for sealing an opening of the bottle. The cap body and the bottle cooperate to define a fermentation space.

The detecting unit is disposed at the cap body, and is configured to continuously monitor at least one environment parameter in the fermentation space by detecting a value of the at least one environment parameter.

The processor is disposed at the cap body and is coupled to the detecting unit. The processor is operable, in response to receipt of an activation signal, to:

estimate when the fermentation process is to be completed, based on the value of the at least one environment parameter detected by the processor;

determine whether the value of the at least one environment parameter is within a predetermined range; and

generate an alert response when it is determined that the value of the at least one environment parameter is out of the predetermined, range.

Another object of the disclosure is to provide a method for monitoring a fermentation process occurring in a bottle, the method to be implemented by a fermentation monitoring device that seals an opening of the bottle and that includes a processor and a detecting unit. The method includes the steps of:

a) continuously detecting, by the detecting unit, a value of at least one environment parameter in a fermentation space that is defined cooperatively by the fermentation monitoring device and the bottle;

b) estimating, by the processor, when the fermentation process is to be completed based on the value of the at least one environment parameter detected in step a);

c) determining, by the processor, whether the value of the at least one environment parameter detected in step a) is within a predetermined range; and

d) generating, by the processor, an alert response when it is determined that the value of the at least one environment parameter is out of the predetermined range.

### BRIEF DESCRIPTION OF THE DRAWINGS

Other features and advantages of the disclosure will become apparent in the following detailed description of the embodiments with reference to the accompanying drawings, of which;

FIG. 1 illustrates a fermentation monitoring device according to one embodiment of the disclosure;

FIG. 2 is a top view of the fermentation monitoring device according to one embodiment of the disclosure;

FIG. 3 is a block diagram illustrating components of the fermentation monitoring device according to one embodiment of the disclosure; and

FIG. 4 is a flow chart illustrating steps of a method for monitoring a fermentation process, according to one embodiment of the disclosure.

### DETAILED DESCRIPTION

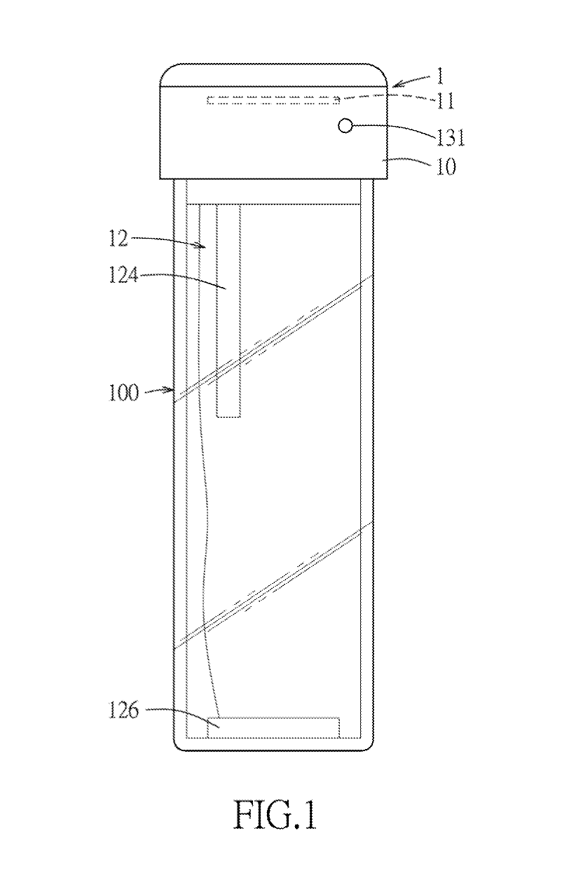

FIG. 1 illustrates a fermentation monitoring device 1 according to one embodiment of the disclosure. The fermentation monitoring device 1 may be embodied using a bottle cap that is made to seal an opening of a bottle 100. As shown in FIG. 1, when the fermentation monitoring device 1 seals the opening of the bottle 100, the fermentation monitoring device 1 and the bottle 100 cooperatively define a fermentation space.

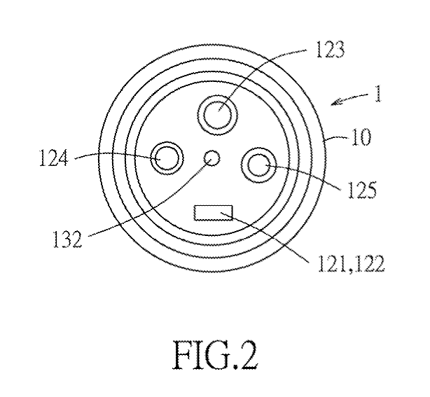

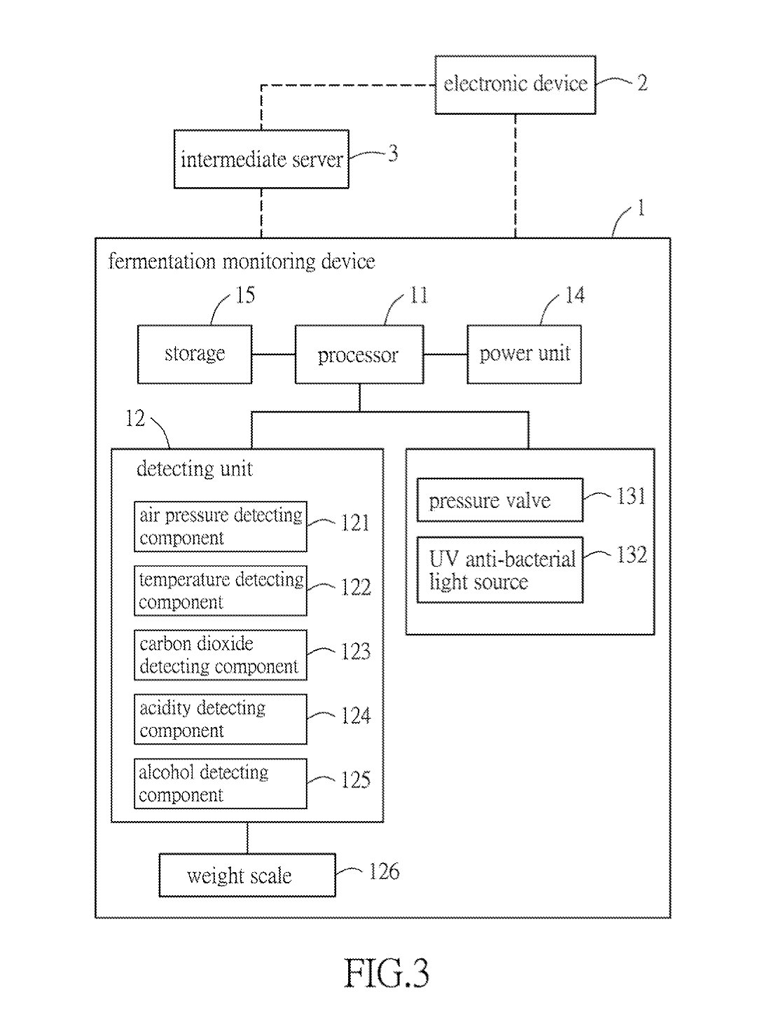

The fermentation monitoring device 1 includes a cap body 10, a processor 11 disposed at the cap body 10, a detecting unit 12, a weight scale 126, a pressure valve 131, an ultraviolet (UV) anti-bacterial light source 132 (see FIG. 2), a power unit 14 (see FIG. 3), and a storage 15.

The detecting unit 12 is coupled to the processor 11, and includes a plurality of detecting components for respectively monitoring a plurality of environment parameters by detecting values of the environment parameters.

Referring to FIG. 3, in this embodiment, the detecting unit 12 includes an air pressure detecting component 121 for detecting a value of an air pressure in the fermentation space, a temperature detecting component 122 for detecting a value of a temperature in the fermentation space, a carbon dioxide detecting component 123 for detecting a value of a carbon dioxide concentration in the fermentation space, an acidity detecting component 124 for detecting a value of an acidity of a solution in the fermentation space, and an alcohol detecting component 125 for detecting a value of an alcohol concentration in the fermentation space. The acidity detecting component 124 extends from the cap body 10 into the fermentation space (as best depicted in FIG. 1), and the other detecting components may be disposed in the cap body 10 and become exposed, as seen in. FIG. 2 (which is a top view of the fermentation monitoring device 1).

The pressure control valve 131 is disposed at the cap body 10, and may be controlled to open or close in order to control the air pressure in the fermentation space.

The UV anti-bacterial light source 132 is disposed at the cap body 10, and may be controlled to activate in order to irradiate the fermentation space.

The power unit 14 provides the power required for operations of the other components of the fermentation monitoring device 1. In this embodiment, the power unit 14 may be recharged wirelessly via an external power source.

The storage 15 is coupled to the processor 11, and stores information regarding the environment parameters therein. Specifically, the storage 15 stores a predetermined range associated with each of the environment parameters.

The processor 11 may be capable of communicating with an electronic device 2, and controlled by the electronic device 2 to perform various operations. The communication between the processor 11 and the electronic device 2 may be done using a local area network (LAN), a Bluetooth® communication, or via an intermediate server 3. The electronic device 2 may be embodied using a smartphone, a laptop computer, a tablet computer, etc.

The processor 11 maybe operable to implement a method for monitoring a fermentation process that is to be carried out in the bottle 100 (i.e., the fermentation space). That is to say, when a user intends to produce a specific product via the fermentation process, the user may place the associated ingredients inside the bottle 100, seal the opening of the bottle 100 using the fermentation monitoring device 1, and control the processor 11 to start monitoring the fermentation process.

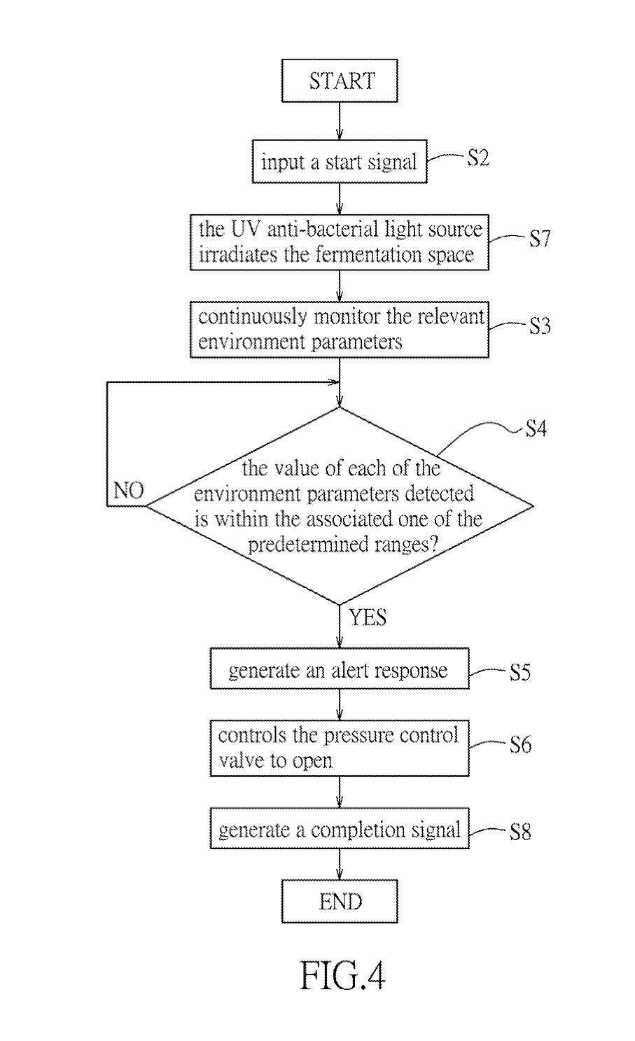

FIG. 4 illustrates steps of a method for monitoring the fermentation process, according to one embodiment of the disclosure.

Before the fermentation process begins, the user may operate the electronic device 2 to transmit a signal to the processor 11. Specifically, the user may operate the electronic device 2 to execute an application.

The application may control the electronic device 2 to display a menu having a plurality of products. Subsequently, the electronic device 2 allows the user to select one of the products that is intended to be produced by the fermentation process. After the user selects one of the products, the electronic device 2 may further display a recipe for the selected one of the products. The recipe may include one or more ingredients, a weight associated with the one or more ingredients, and, in the case that a plurality of ingredients are involved, a specific order in which the ingredients are to be put into the bottle 100. The weight scale 126 is disposed at a bottom surface of the bottle 100 and coupled to the 11 processor for detecting a weight of content in the bottle 100. After the ingredient(s) is (are) put in the bottle 100, the fermentation monitoring device 1 is used to seal the bottle 100 thereby defining the fermentation space.

To initiate the method for monitoring the fermentation process, in step S2, the user operates the electronic device 2 to input a start signal. In response, the electronic device 2 communicates with the fermentation monitoring device 1 and transmits the start signal to the fermentation monitoring device 1, indicating that monitoring of the fermentation process is to be started. Based on the user input of the product, the processor 11 of the fermentation monitoring device may determine which ones of the environment parameters are to be monitored, and adjust the predetermined ranges associated with the to-be-monitored ones of the environment parameters based on the user input of the product so that the predetermined ranges is appropriate for the product.

In step S3, the detecting unit 12 is activated to continuously monitor the relevant environment parameters.

Moreover, the processor 11 estimates when the fermentation process is to be completed, based on the values of the environment parameters that are most recently detected. In this embodiment, the processor 11 estimates a time to completion to indicate when the fermentation process is to be completed, and transmits a notification of the time to completion to the electronic device 2 so as to enable the electronic device 2 to count down the time to completion.

It is worth noting that in one embodiment, the processor 11 periodically estimates a time to completion. That is done because the values of the environment parameters in the fermentation space may change rapidly during the fermentation process, and by dynamically estimating and updating the time to completion, a more accurate estimation may yield. In some cases, when the value(s) of one or more particular environment parameters (e.g., a concentration of alcohol) reaches a specific level, the processor 11 may determine that the fermentation process is complete.

In step S4, during the fermentation process, the processor 11 determines whether the value of each of the environment parameters detected in step S3 is within the associated one of the predetermined ranges.

When it is determined that the value of one of the monitored environment parameters is out of the associated one of the predetermined ranges, the processor 11 generates an alert response in step S5. The alert response may be transmitted to the electronic device 2 so as to notify the user, such that the user may perform various procedures to rectify the environment parameter(s).

The processor 11 may automatically address some of the issues associated with the environment parameter (s) and determined in step S4. For example, in step S6, when it is determined that the air pressure in the fermentation space is larger than an upper limit of the associated predetermined range, the processor 11 controls the pressure control valve 131 to open so as to decrease the air pressure in the fermentation space.

It is noted that, during any stage of the fermentation process, the UV anti-bacterial light source 132 may be activated by the processor 11 in order to irradiate the fermentation space (step S7). In this embodiment, the UV anti-bacterial light source 132 may be activated right after the monitoring of the fermentation process has started.

In step S8, when the time to completion estimated by the processor 11 becomes zero and/or the processor 11 determines that the fermentation process is complete, the processor 11 may generate a completion signal and transmit the completion signal to the electronic device 2. Alternatively, when the countdown of the time to completion conducted by the electronic device 2 reaches zero, the electronic device 2 generates an alert to notify the user that the fermentation process has completed.

To sum up, the fermentation device 1 and the method for monitoring the fermentation process as described in the disclosure are configured to monitor the environment parameters in the fermentation space, and to provide an alert response when the values of the environment parameters is out of the respective predetermined ranges. Moreover, the fermentation device 1 is configured to estimate the time to completion and to address some of the issues regarding the environment parameters automatically, and is therefore able to provide the user with a more user-friendly environment for executing the fermentation process.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A fermentation monitoring device for monitoring a fermentation process occurring in a bottle, said fermentation monitoring device comprising:
a cap body for sealing an opening of the bottle, said cap body and the bottle cooperating to define a fermentation space;
a detecting unit disposed at said cap body, and configured to continuously monitor at least one environment parameter in the fermentation space by detecting a value of the at least one environment parameter; and
a processor disposed at said cap body and coupled to said defecting unit, said processor being operable, in response to receipt of an activation signal, to estimate when the fermentation process is to be completed, based on the value of the at least one environment parameter detected by said detecting unit;
determine whether the value of the at least one environment parameter is within a predetermined range; and
generate an alert response when it is determined that the value of the at least one environment parameter is out of the predetermined range.

2. The fermentation monitoring device of claim 1, wherein the at least one environment parameter that is monitored by said detecting unit includes one of an air pressure in the fermentation space, a temperature in the fermentation space, a carbon dioxide concentration in the fermentation space, an acidity of a solution in the fermentation space, an alcohol concentration in the fermentation space and combinations thereof.

3. The fermentation monitoring device of claim 1, wherein said detecting unit includes an acidity detecting component that extends from said cap body into the fermentation space for detecting a value of an acidity of a solution in the fermentation space.

4 . The fermentation monitoring device of claim 1, further comprising a weight scale that is to be disposed at a bottom surface of the bottle and. coupled to said processor for detecting a weight of content in the bottle.

5. The fermentation monitoring device of claim 1, wherein said processor is operable, in response to receipt of a user input of a product that is intended to be produced by the fermentation process, to adjust the predetermined range based on the user input of the product.

6 . The fermentation monitoring device of claim 1, further comprising a pressure control valve that is coupled to and controlled by said processor, wherein:
the at least one environment parameter monitored by said detecting unit includes an air pressure in the fermentation space; and
when it is determined that the value of the air pressure in the fermentation space is larger than an upper limit of the predetermined range, said processor is configured to control said pressure control valve to open so as to decrease the value of the air pressure in the fermentation space.

7 . The fermentation monitoring device of claim 1, further comprising an ultraviolet (UV) anti-bacterial light source coupled to said processor, and controlled by said processor to activate for irradiating the fermentation space.

8 . The fermentation monitoring device of claim 1, wherein said processor is capable of communicating with an electronic device, and is programmed to:
periodically estimate a time to completion to indicate when the fermentation process is to be completed based on the value of the at least one environment parameter that is most recently detected;
transmit a notification of the time to completion to the electronic device so as to enable the electronic device to count down the time to completion.

9. A method for monitoring a fermentation process occurring in a bottle, the method to be implemented by a fermentation monitoring device that seals an opening of the bottle and that includes a processor and a detecting unit, the method comprising the steps of:
a) continuously detecting, by the detecting unit, a value of at least one environment parameter in a fermentation space that is defined cooperatively by the fermentation monitoring device and the bottle;
b) estimating, by the processor, when the fermentation process is to be completed based on the value of the at least one environment parameter detected in step a);
c) determining, by the processor, whether the value of the at least one environment parameter detected in step a) is within a predetermined range; and
d) generating, by the processor, an alert response when it is determined that the value of the at least one environment parameter is out of the predetermined range during the fermentation process.

10. The method of claim 9, the processor of the fermentation monitoring device being capable of communicating with an electronic device, wherein the fermentation monitoring device is operable to implement the method in response to receipt of an activation signal from the electronic device, and in step d), the alert response is transmitted to the electronic device.

11. The method of claim 9, the processor of the fermentation monitoring device being capable of communicating with an electronic device, wherein in step b), the processor is programmed to:
periodically estimate a time to completion to indicate when the fermentation process is to be completed based on the value of the at least one environment parameter that is most recently detected in step a);
transmit a notification of the time to completion to the electronic device so as to enable the electronic device to count down the time to completion.

12. The method of claim 9, further comprising, before step a), the step of, in response to receipt of a user input of a product that is intended to be produced by the fermentation process, adjusting the predetermined range based on the user input of the product.

13. The method of claim 9, the fermentation monitoring device further including a pressure control valve,
wherein, in step a), the at least one environment parameter includes an air pressure in the fermentation space; and
wherein the method further comprises the step of, when it is determined that the value of the air pressure in the fermentation space is larger than an upper limit of the predetermined range, opening, by the processor, the pressure control valve so as to decrease the value of the air pressure in the fermentation space.

14. The method of claim 9, the fermentation monitoring device further including an ultraviolet (UV) anti-bacterial light source,
wherein the method further comprises the step of activating, by the processor, the UV anti-bacterial light source for irradiating the fermentation space.

15. The method of claim 9, wherein in step a), the at least one environment parameter includes one of an air pressure in the fermentation space, a temperature in the fermentation space, a carbon dioxide concentration in the fermentation space, an acidity of a solution in the fermentation space, an alcohol concentration in the fermentation space and combinations thereof.

16. The method of claim 9, the method to be implemented further by an electronic device capable of communicating with the fermentation monitoring device, wherein the method further comprises the steps of:
displaying, by the electronic device, a menu having a plurality of products and allowing a user to select one of the products that is intended to be produced by the fermentation process; and
displaying a recipe for a selected one of the products.
